(19) United States
(12) Patent Application Publication
(10) Pub. No.: US 2003/0115634 A1
(43) Pub. Date: Jun. 19, 2003

(54) Genes of the 1-desoxy -d-xylulose biosynthesis path

(76) Inventor: Hassan Jomaa, Giessen (DE)

Correspondence Address: WILLIAM NEWELL SHERIDAN, 945 MOUNTAIN BRANCH DRIVE, BIRMINGHAM, AL, 35226, US

(21) Appl. No.: 10/275,360

(22) PCT Filed: Apr. 21, 2001
(86) PCT No.: PCT/EP01/04537

(30) Foreign Application Priority Data
May 5, 2000 (DE) 100 21 688.9

(51) Int. Cl. A01H 1/00; C07H 21/04; C12N 9/16; C12N 7/00; C12N 15/82; C12N 15/74; C12P 21/02; C12N 1/21

(52) U.S. Cl. 800/284; 435/69.1; 435/196; 435/320.1; 435/419; 435/235.1; 435/252.3; 536/23.2

(57) ABSTRACT

The invention relates to DNA sequences from Plasmodium falciparum, namely the genes lytB and yfgB which, when integrated into the genome of viruses, eukaryotes and prokaryotes, alter the isoprenoid biosynthesis. The invention also relates to gene technological methods for producing these transgenic viruses, eukaryotes and prokaryotes and to methods for identifying substances with a herbicidal, antimicrobial, antiparasitic, antiviral, fungicidal and bactericidal effect in plants and an antimicrobial, antiparasitic, antimycotic, antibacterial and antiviral effect in human beings and animals.

The present invention relates to DNA sequences which modify isoprenoid synthesis when integrated into the genome of viruses, eukaryotes and prokaryotes and to genetic engineering processes for the production of these transgenic viruses, eukaryotes and prokaryotes. It also relates to methods for the identification of substances having a herbicidal, antimicrobial, antiparasitic, antiviral, fungicidal or bactericidal action in plants or an antimicrobial, antiparasitic, antimycotic, antibacterial or antiviral action in humans and animals.

The biosynthesis pathway for the formation of isoprenoids via the conventional acetate/mevalonate pathway and an alternative mevalonate-independent biosynthesis pathway, the deoxy-D-xylulose phosphate pathway (DOXP or MEP pathway) are already known (Rohmer, M., Knani, M., Simonin, P., Sutter, B., and Sahm, H. (1993): Biochem. J. 295: 517-524).

However, how and via what routes a change in the isoprenoid concentration can be achieved via the deoxy-D-xylulose phosphate pathway in viruses, eukaryotes and prokaryotes is not known.

DNA sequences which code for enzymes which participate in the DOXP pathway are therefore provided. Both genes (lytB and yfgB) and enzymes (LytB and YfgB) participate in isoprenoid biosynthesis and are essential for the survival of the particular organisms (example 1 and 2).

The invention relates to the following DNA sequences:

DNA sequences which code for a polypeptide with the amino acid sequence shown in SEQ ID NO: 5 or for an analogue or derivative of the polypeptide according to SEQ ID NO: 5 wherein one or more amino acids have been deleted, added or replaced by other amino acids, without substantially reducing the enzymatic action of the polypeptide, and

DNA sequences which code for a polypeptide with the amino acid sequence shown in SEQ ID NO: 14 or for an analogue or derivative of the polypeptide according to SEQ ID NO: 14 wherein one or more amino acids have been deleted, added or replaced by other amino acids, without substantially reducing the enzymatic action of the polypeptide.

The invention is furthermore defined by claims 1 to 4. Further developments of the invention are defined by the sub-claims.

The genes and their gene products (polypeptides) are listed in the sequence listing with their primary structure and have the following allocation:

SEQ ID NO: 1: lytB gene

SEQ ID NO: 5: LytB protein

SEQ ID NO: 9: yfgB gene

SEQ ID NO: 14: YfgB protein

The DNA sequences all originate from Plasmodium falciparum, strain 3D7.

In addition to the DNA sequences mentioned in the sequence listing, those which have a different DNA sequence as a result of degeneration of the genetic code but code for the same polypeptide or for an analogue or derivative of the polypeptide wherein one or more amino acids have been deleted, added or replaced by other amino acids, without substantially reducing the enzymatic action of the polypeptide, are also suitable.

The sequences according to the invention are suitable for over-expression of genes in viruses, eukaryotes and prokaryotes which are responsible for isoprenoid biosynthesis of the 1-deoxy-D-xylulose pathway.

According to the invention, animal cells, plant cells, algae, yeasts and fungi belong to the eukaryotes or eukaryotic cells, and archaebacteria and eubacteria belong to the prokaryotes or prokaryotic cells.

When a DNA sequence on which one of the abovementioned DNA sequences is located is integrated into a genome, expression of the genes described above in viruses, eukaryotes and prokaryotes becomes possible. The viruses, eukaryotes and prokaryotes transformed according to the invention are cultured in a manner known per se and the isoprenoid formed during this culturing is isolated and optionally purified. Not all the isoprenoids have to be isolated, since in some cases the isoprenoids are released directly into the surrounding air.

The invention furthermore relates to a process for the production of transgenic viruses, eukaryotes and prokaryotes with isoprenoid expression, which comprises the following steps.

a) Preparation of a DNA sequence with the following part sequences

i) promoter which is active in viruses, eukaryotes and prokaryotes and ensures the formation of an RNA in the envisaged target tissue or the target cells,

ii) DNA sequence which codes for a polypeptide with the amino acid sequence shown in SEQ ID NO: 5 or 14 or for an analogue or derivative of the polypeptide according to SEQ ID NO: 5 or 14,

iii) 3′-nontranslated sequence which leads to the addition of poly-A radicals on to the 3′-end of the RNA in viruses, eukaryotes and prokaryotes,

b) transfer and incorporation of the DNA sequence into the genome of viruses or prokaryotic or eukaryotic cells with or without the use of a vector (e.g. plasmid, viral DNA).

The intact whole plants can be regenerated from the transformed plant cells.

The sequences with the nucleotide sequences SEQ ID NO: 1 and SEQ ID NO: 9 which code for the proteins can be provided with a promoter which ensures transcription in particular organs or cells and is coupled in the sense orientation (3′-end of the promoter to the 5′-end of the coding sequence) to the sequence which codes the protein to be formed. A termination signal which determines the termination of the mRNA synthesis is attached to the 3′-end of the coding sequence. To direct the protein to be expressed into particular subcellular compartments, such as chloroplasts, amyloplasts, mitochondria, vacuoles, cytosol or intercellular spaces, a sequence which codes for a so-called signal sequence or a transit peptide can also be placed between the promoter and the coding sequence. The sequence must be in the same reading frame as the coding sequence of the protein. For preparation of the introduction of the DNA sequences according to the invention into higher plants, a large number of cloning vectors which comprise a replication signal for E. coli and a marker which allows selection of the transformed cells are available. Examples of vectors are pBR 322, pUC series, M13mp series, pACYC 184, EMBL 3 etc. Further DNA sequences may be required, depending on the method of introduction of desired genes into the plants. For example, if the Ti or Ri plasmid is used for transformation of the plant cells, at least a right limitation, but often the right and the left limitation of the Ti and Ri plasmid T-DNA must be inserted as a flanking region to the genes to be introduced. The use of T-DNA for transformation of plant cells has been investigated intensively and has been described adequately in EP 120516; Hoekama, in: The Binary Plant Vector System, Offset-drukkerij Kanters B. V. Alblasserdam (1985), Chapter V; Fraley et al., Crit.Rev.Plant Sci. 4, 1-46 and An et al. (1985) EMBO J. 4, 277-287. Once the DNA introduced has integrated into the genome, it is as a rule stable and is also retained in the descendants of the cells originally transformed. It usually contains a selection marker, which imparts to the transformed plant cells resistance to a biocide or an antibiotic, such as kanamycin, G 418, bleomycin, hygromycin or phosphinotricin, inter alia. The marker individually used should therefore allow selection of transformed cells over cells in which the DNA inserted is missing.

Many techniques are available for introduction of DNA into a plant. These techniques include transformation with the aid of agrobacteria, e.g. Agrobacterium tumefaciens, fusion of protoplasts, microinjection of DNA, electroporation, as well as ballistic methods and virus infection. Whole plants can then be regenerated again from the transformed plant material in a suitable medium, which can contain antibiotics or biocides for selection. No specific requirements are imposed on the plasmids for the injection and electroporation. However, if whole plants are to be regenerated from cells transformed in this way, the presence of a selectable marker gene is necessary. The transformed cells grow within the plants in the usual way (McCormick et al. (1986), Plant Cell Reports 5, 81-84). The plants can be grown normally and crossed with plants which have the same transformed genetic disposition or other genetic dispositions. The individuals arising therefrom have the corresponding phenotypic characteristics.

The invention also provides expression vectors which contain one or more of the DNA sequences according to the invention. Such expression vectors are obtained by providing the DNA sequences according to the invention with suitable functional regulation signals. Such regulation signals are DNA sequences which are responsible for the expression, for example promoters, operators, enhancers and ribosomal binding sites, and are recognized by the host organism.

Further regulation signals, which control, for example, replication or recombination of the recombinant DNA in the host organism, can optionally also be a constituent of the expression vector.

The invention also provides the host organisms transformed with the DNA sequences or expression vectors according to the invention.

Those host cells and organisms which have no intrinsic enzymes of the DOXP pathway are particularly suitable for expression of the enzymes according to the invention. This applies to archaebacteria, animals, some fungi, slime fungi and some eubacteria. The detection and purification of the recombinant enzymes is substantially facilitated by the absence of these intrinsic enzyme activities. It is also possible for the first time, as a result, to measure the activity and in particular the inhibition of the activity of the recombinant enzymes according to the invention by various chemicals and pharmaceuticals in crude extracts from the host cells with a low outlay.

The expression of the enzymes according to the invention advantageously then takes place in eukaryotic cells if posttranslatory modifications and a natural folding of the polypeptide chain are to be achieved. Depending on the expression system, expression of genomic DNA sequences moreover has the result that introns are eliminated by splicing the DNA and the enzymes are produced in the polypeptide sequence characteristic for the parasites. Sequences which code for introns can also be eliminated from the DNA sequences to be expressed or inserted experimentally by recombinant DNA technology.

The protein can be isolated from the host cell or the culture supernatant of the host cell by processes known to the expert. In vitro reactivation of the enzymes may also be necessary.

To facilitate the purification, the enzymes according to the invention or part sequences of the enzymes can be expressed as a fusion protein with various peptide chains. Oligo-histidine sequences and sequences which are derived from glutathione S-transferase, thioredoxin or calmodulin-binding peptides are particularly suitable for this purpose. Fusions with thioredoxin-derived sequences are particularly suitable for prokaryotic expression, since the solubility of the recombinant enzymes is increased as a result.

The enzymes according to the invention or part sequences of the enzymes can furthermore be expressed as a fusion protein with those peptide chains known to the expert, such that the recombinant enzymes are transported into the extracellular medium or into particular compartments of the host cells. Both the purification and the investigation of the biological activity of the enzymes can be facilitated as a result.

In the expression of the enzymes according to the invention, it may prove to be expedient to modify individual codons. Targeted replacement of bases in the coding region is also appropriate here if the codons used deviate in the parasites from the codon utilization in the heterologous expression system, in order to ensure optimum synthesis of the protein. Deletions of non-translated 5′- or 3′-sections are furthermore often appropriate, for example if several destabilizing sequence motifs ATTTA are present in the 3′-region of the DNA. These should then be deleted in the case of the preferred expression in eukaryotes. Modifications of this type are deletions, additions or replacement of bases, and the present invention also provides these.

The enzymes according to the invention can furthermore be obtained by in vitro translation under standardized conditions by techniques known to the expert. Systems which are suitable for this are rabbit reticulocyte and wheat germ extracts and bacterial lysates. Translation of in vitro-transcribed mRNA in Xenopus oocytes is also possible.

Oligo- and polypeptides with sequences derived from the peptide sequence of the enzymes according to the invention can be prepared by chemical synthesis. With suitable choice of the sequences, such peptides have properties which are characteristic of the complete enzymes according to the invention. Such peptides can be prepared in large amounts and are particularly suitable for studies of the kinetics of the enzyme activity, the regulation of the enzyme activity, the three-dimensional structure of the enzymes, the inhibition of the enzyme activity by various chemicals and pharmaceuticals and the binding geometry and binding affinity of various ligands.

A DNA with the nucleotides from sequences SEQ ID NO: 1 and 9 is preferably used for recombinant preparation of the enzymes according to the invention.

As stated above, in addition to the conventional acetate/mevalonate pathway, there is an alternative mevalonate-independent biosynthesis pathway in plants for the formation of isoprenoids, the deoxy-D-xylulose phosphate pathway (DOXP pathway). It has emerged that this deoxy-D-xylulose phosphate metabolic pathway is also present in many parasites, bacteria, viruses and fungi.

The invention therefore also includes a method for screening a compound. According to this method, a host organism which contains a recombinant expression vector, wherein the vector has at least part of the oligonucleotide sequence according to SEQ ID NO: 1 or SEQ ID NO: 9 or variants or homologues of this, and in addition a compound which is presumed to have an antimicrobial, antiparasitic, antiviral and antimycotic action in humans and animals or a bactericidal, antimicrobial, herbicidal or fungicidal action in plants are provided. The host organism is then brought into contact with the compound and the activity of the compound is determined.

This invention also provides methods for the determination of the enzymatic activity of the LytB and YfgB protein. This can be determined by the known techniques. In these, the change in the concentration of the intermediates of the DOXP pathway which function as substrates or products of the particular enzymes is determined by photometric, fluorimetric or chromatographic methods. The detection of the change in concentration can also be carried out by coupled enzyme assays, the detection taking place via one or more additional enzymatic steps. The additional enzymes may also participate in the DOXP pathway or can be added experimentally to the system.

### EXAMPLE 1

This investigate whether the lytB gene product is necessary for the survival of the blood stages of the malaria pathogen Plasmodium falciparum, production of a “gene disruption” mutant of P. falciparum was attempted. In this mutant, a gene which codes for a selection marker was to be introduced into the gcpe gene by genetic engineering methods, and this was to be inactivated as a result. For this, a construct (pPflytBKO) which contains an expression cassette which imparts pyrimethamine resistance and is flanked by two fragments from the coding sequence of the lytB gene of P. falciparum was produced. This construct was to be integrated into the gcpe gene by homologous recombination via the flanking sequences.

All the PCR amplifications described were carried out with heat-stable Pwo DNA polymerase, as a result of which the products acquire smooth ends and are suitable for “blunt end” ligations. The sequence of the lytB gene was amplified with the primers 5′-ATG TCA GTT ACC ACA TTT TGT TCT TTA AAA AAA ACG G-3′ and 5′-GTG ATT TCA TTT TTC TCT TTC TTT TAT CAT C-3′ and genomic DNA from the P. falciparum strain 3D7 as the template, phosphorylated with T4 polynucleotide kinase and cloned into a pUC 19 vector linearized with Sma I (pUCPflytB). The dihydrofolate reductase gene of Toxoplasma gondii (Tg DHFR-TS), which had been modified such that it imparts resistance to pyrimethamine, was used as the selection marker. The expression of TgDHFR-TS took place under the control of the 5′- and 3′-nontranslated elements of the P. falciparum calmodulin (Pf CAM) gene. This expression cassette was obtained from the plasmid pTgD-TS.CAM5/3.KP, which had been constructed according to published protocols (Crabb, B. S. and Cowman, A. F. (1996) Proc. Natl. Acad. Sci. USA, 93, 7289-7294). The expression cassette was obtained by amplification with the primers 5′-AATCTCTGAGCTTCTTCTTTG-3′ and 5′-GGGGGAGCTCGAACTTAATAAAAAAGAGGAG-3′ with pTgD-TS.CAM5/3.KP as the template. The expression cassette was then inserted into the insert of pUCPfgcpe. For this, pUCPflytB was opened with Dsa I in the insert and the overhangs were completed with T4 and Klenow DNA polymerase. The amplified expression cassette was phosphorylated and inserted via “blunt end” ligation, as a result of which pPflytBKO was obtained.

For transfection by electroporation, the infected erythrocytes (strain 3D7, chiefly ring stages, approx. 15% parasitaemia) of a 10 cm culture dish were pelleted and resuspended in 0.8 ml Cytomix (120 mM KCl; 0.15 mM CaCl₂; 2 mM EGTA; 5 mM MgCl₂; 10 mM K₂HPO₄/KH₂PO₄; 25 mM HEPES, pH 7.6), which contained 150 μg plasmid DNA from pPflytBKO. The electroporation was carried out in 4 mm cells at 2.5 kV, 200 Ohm and 25 μF. The parasites were then plated out again on culture dishes and incubated. 48 h after the transfection 400 nM pyrimethamine was added to the culture medium, and after a further 48 h the pyrimethamine concentration was reduced to 100 nM. After 22 days it was possible to detect resistant parasites under the microscope. After 6 weeks the pyrimethamine concentration was increased to 2 μM for a further 3 weeks. The parasites were cloned by limiting dilution on 96-well cell culture plates and cultured for 11 days in the absence of pyrimethamine. 1 μM pyrimethamine was then added again. Episomal plasmids are lost by culture in the absence of pyrimethamine, and during the subsequent renewed selection only parasites which have integrated the plasmid chromosomally can survive.

Parasites grew in only 5 wells, since the plasmid evidently was present episomally in most of the parasites. It was still possible to detect expression of the lytB gene by RT-PCR in these clones. The plasmid was thus integrated into the genome by non-homologous recombination and the lytB gene of the parasites was not inactivated. Parasites with an inactivated lytB gene are thus evidently not viable, and the gene is therefore essential. According to recent findings, the genus Plasmodium is phylogenetically close to lower algae (Fichera, M. E. and Roos, D. S. (1997) Nature, 390, 407-409; Köhler, S, Delwiche, C. F., Denny, P. W., Tilney, L. G., Webster, P., Wilson, R. J. M., Palmer, J. D. and Roos, D. S. (1997) Nature, 275, 1485-1489). It is therefore to be deduced that the lytB gene is evidently also essential for plants.

### EXAMPLE 2

To investigate whether the yfgB gene product is necessary for the survival of the blood stages of the malaria pathogen Plasmodium falciparum, production of a “gene disruption” mutant of P. falciparum was attempted. In this mutant, a gene which codes for a selection marker was to be introduced into the yfgB gene by genetic engineering methods, and this was to be inactivated as a result. For this, a construct (pPfyfgBKO) which contains an expression cassette which imparts pyrimethamine resistance and is flanked by two fragments from the coding sequence of the yfgB gene of P. falciparum was produced. This construct was to be integrated into the gcpe gene by homologous recombination via the flanking sequences.

All the PCR amplifications described were carried out with heat-stable Pwo DNA polymerase, as a result of which the products acquire smooth ends and are suitable for “blunt end” ligations. The yfgB sequence was amplified with the primers 5′-ATG GAA AAG TCA AAA AGG TAC ATA AGC CTG-3′ and 5′-AGC ATC GTC CAA ACG ATG AAA ATT TTC GTC-3′ and genomic DNA from the P. falciparum strain 3D7 as the template, phosphorylated with T4 polynucleotide kinase and cloned into a pUC 19 vector linearized with Sma I (pUCPfyfgB). The dihydrofolate reductase gene of Toxoplasma gondii (Tg DHFR-TS), which had been modified such that it imparts resistance to pyrimethamine, was used as the selection marker. The expression of TgDHFR-TS took place under the control of the 5′- and 3′-nontranslated elements of the P. falciparum calmodulin (Pf CAM) gene. This expression cassette was obtained from the plasmid pTgD-TS.CAM5/3.KP, which had been constructed according to published protocols (Crabb, B. S. and Cowman, A. F. (1996) Proc. Natl. Acad. Sci. USA, 93, 7289-7294). The expression cassette was obtained by amplification with the primers 5′-AATCTCTGAGCTTCTTCTTTG-3′ and 5′-GGGGGAGCTCGAACTTAATAAAAAAGAGGAG-3′ with pTgD-TS.CAM5/3.KP as the template. The expression cassette was then inserted into the insert of pUCPfyfgB. For this, pUCPfgcpe was opened with Pac I in the insert and the overhangs were completed with T4 and Klenow DNA polymerase. The amplified expression cassette was phosphorylated and inserted via “blunt end” ligation, as a result of which pPfyfgBKO was obtained.

For transfection by electroporation, the infected erythrocytes (strain 3D7, chiefly ring stages, approx. 15% parasitaemia) of a 10 cm culture dish were pelleted and resuspended in 0.8 ml Cytomix (120 mM KC1; 0.15 mM CaCl₂; 2 mM EGTA; 5 mM MgCl₂; 10 mM K₂HPO₄/KH₂PO₄; 25 mM HEPES, pH 7.6), which contained 150 μg plasmid DNA from pPfyfgBKO. The electroporation was carried out in 4 mm cells at 2.5 kV, 200 Ohm and 25 μF. The parasites were then plated out again on culture dishes and incubated. 48 h after the transfection 400 nM pyrimethamine was added to the culture medium, and after a further 48 h the pyrimethamine concentration was reduced to 100 nM. After 18 days it was possible to detect resistant parasites under the microscope. After 6 weeks the pyrimethamine concentration was increased to 2 μM for a further 3 weeks. The parasites were cloned by limiting dilution on 96-well cell culture plates and cultured for 11 days in the absence of pyrimethamine. 1 μM pyrimethamine was then added again. Episomal plasmids are lost by culture in the absence of pyrimethamine, and during the subsequent renewed selection only parasites which have integrated the plasmid chromosomally can survive. None of the parasite clones survived the renewed addition of pyrimethamine. This result indicates that parasites with an inactivated yfgB gene are not viable, and the gene is therefore essential. According to recent findings, the genus Plasmodium is phylogenetically close to lower algae (Fichera, M. E. and Roos, D. S. (1997) Nature, 390, 407-409; Köhler, S, Delwiche, C. F., Denny, P. W., Tilney, L. G., Webster, P., Wilson, R. J. M., Palmer, J. D. and Roos, D. S. (1997) Nature, 275, 1485-1489). It is therefore to be deduced that the yfgB gene is evidently also essential for plants.

### EXAMPLE 3

### The yfgB is Essential for Escherichia Coli

Construction of the gene replacement plasmid pKO3-ΔyfgB

The pKO3 vector was used to produce a deletion mutant of E. coli (Link, A. J.; Phillips, D.; Church, G. M.; J. Bacteriol. 179, 6228-6237). To produce the deletion construct, two sequences downstream and upstream of the yfgB gene were amplified in two asymmetric PCR batches. The primers were employed in a 1:10 molar ratio (50 nM and 500 nM). The two PCR products were fused to one product in a second PCR amplification. The product was cloned using the pCR-TA-TOPO Cloning Kit (Invitrogen) and cloned into the pKO3 vector via the restriction cleavage sites Bam HI and Sal I. The following primers were used:

yfgB-N-out,
5′-AGGATCCtccatcatcaaaccgaac-3′
yfgB-N-in,
5′-TCCCATCCACTAAACTTAAACATctattccggcctcgttat-3′
yfgB-C-in,
5′-ATGTTTAAGTTTAGTGGATGGGaagcggtctgatagccatt-3′
yfgB-C-out,
5′-AGTCGACaagtggagcctgcttttc-3′.

The restriction cleavage sites are underlined. Overlapping sequences which define a 21 bp “in frame” insertion are printed in bold.

Construction of the Deletion Mutant wtΔyfgB

The “gene replacement” experiments were carried out in a manner similar to that described (Link, A. J.; Phillips, D.; Church, G. M.; J. Bacteriol. 179, 6228-6237). The plasmid pKO3-ΔyfgB was transformed into the E. coli K-12 strain DSM No. 498 (ATCC 23716). After incubation for 1 h at 30° C., bacteria with integrated plasmid were selected by a temperature shift to 43° C. By subsequent testing for sucrose resistance and chloramphenicol sensitivity, bacteria which had lost the vector sequences were selected and then analysed for the desired genotype by PCR. No bacteria with a yfgB deletion were to be discovered, which demonstrates that the yfgB gene is essential for E. coli.

1. DNA sequences which code for a polypeptide with the amino acid sequence shown in SEQ ID NO: 5 or for an analogue or derivative of the polypeptide according to SEQ ID NO: 5 wherein one or more amino acids have been deleted, added or replaced by other amino acids, without substantially reducing the enzymatic action of the polypeptide.

2. DNA sequence according to claim 1, with the amino acid sequence shown in SEQ ID NO: 1.

3. DNA sequences which code for a polypeptide with the amino acid sequence shown in SEQ ID NO: 14 or for an analogue or derivative of the polypeptide according to SEQ ID NO: 14 wherein one or more amino acids have been deleted, added or replaced by other amino acids, without substantially reducing the enzymatic action of the polypeptide.

4. DNA sequence according to claim 3, with the amino acid sequence shown in SEQ ID NO: 9.

5. DNA sequence according to one of claims 1 to 4, characterized in that it also has functional regulation signals, in particular promoters, operators, enhancers and ribosomal binding sites.

6. DNA sequence with the following part sequences
i) promoter which is active in viruses, eukaryotes and prokaryotes and ensures the formation of an RNA in the envisaged target tissue or the target cells,
ii) DNA sequence which codes for a polypeptide with the amino acid sequence shown in SEQ ID NO: 5 or 14 or for an analogue or derivative of the polypeptide according to SEQ ID NO: 5 or 14,
iii) 3′-nontranslated sequence which leads to the addition of poly-A radicals on to the 3′-end of the RNA in viruses, eukaryotes and prokaryotes.

7. Expression vector containing one or more DNA sequences according to one of claims 1 to 4.

8. Protein which participates in the 1-deoxy-D-xylulose 5-phosphate metabolic pathway and a) is coded by the DNA sequence SEQ ID NO: 1 or 9 or b) is coded by DNA sequences which hybridize with the DNA sequences SEQ ID NO: 1 or 9 or fragments of these DNA sequences in the DNA region which codes for the mature protein or c) is coded by DNA sequences which would hybridize with the sequences defined in b) without degeneration of the genetic code and code for a polypeptide with a corresponding amino acid sequence.

9. Protein according to claim 8, which has the amino acid sequences SEQ ID NO: 5 or 14.

10. Plant cells containing DNA sequences according to one of claims 1 to 4.

11. Transformed plant cells and transgenic plants regenerated from these containing DNA sequences according to one of claims 1 to 4.

12. Transgenic viruses, eukaryotes and prokaryotes with isoprenoid expression, characterized in that they contain a DNA sequence according to one of claims 1 to 4.

13. Use of a DNA sequence according to one of claims 1 to 4 for determination of the enzymatic activity of the LytB and YfgB protein.

14. Use of a DNA sequence according to one of claims 1 to 4 for modifying, in particular increasing, the isoprenoid content in viruses and eukaryotic and prokaryotic cells.

15. Use of DNA sequences according to one of claims 1 to 4 for identification of substances which have an inhibiting action on the LytB and YfgB protein.

16. Process for isolation of a protein according to claim 8, characterized in that culture supernatants of parasites or of broken-down parasites are purified via chromatographic and electrophoretic techniques.

17. Process for isolation of a protein according to claim 8, characterized in that it is the product of a viral, prokaryotic or eukaryotic expression of an exogenous DNA.

18. Method for determination of the enzymatic activity of the LytB and YfgB protein, characterized in that the change in the concentration of the substrates, co-substrates and products is determined.

19. Process for the production of transgenic viruses, eukaryotes and prokaryotes with isoprenoid expression, characterized in that a DNA sequence according to claim 4 or 5 is transferred and incorporated into the genome of viruses and eukaryotic and prokaryotic cells, with or without the use of a plasmid.

20. Method for screening a compound, wherein the method comprises:
a) provision of a host cell which contains a recombinant expression vector, wherein the vector has at least part of the oligonucleotide sequence according to SEQ ID NO: 1 or SEQ ID NO: 9 or variants or analogues of this, and in addition a compound which is presumed to have an antimycotic, antibiotic, antiparasitic or antiviral action in humans and animals,
b) bringing the microorganism into contact with the compound and
c) determination of the antimycotic, antibiotic, antiparasitic or antiviral activity of the compound.

21. Method for screening a compound, wherein the method comprises:
a) provision of a host cell which contains a recombinant expression vector, wherein the vector has at least part of the oligonucleotide sequence according to SEQ ID NO: 1 or SEQ ID NO: 9 or variants or analogues of this, and in addition a compound which is presumed to have an antimycotic, antibiotic, antiparasitic or antiviral action in humans and animals,
b) bringing the microorganism into contact with the compound and
c) determination of the bactericidal, fungicidal or herbicidal activity of the compound.


SEQUENCE LISTING (28 sequences)

SEQ ID NO: 1 - DNA, 1920 bp, Plasmodium falciparum
  CDS (1)..(1920)
ttatacacatattgaacaaaaaaaaaaaagaaaaaaaaaaaaaaaaaaaaaaaaaaaaaactattaacttatattttttatgtattattatattatctatactttcatttatttatttatttattttattttattttttttatttcccgataacgttatatatatttatatatatatatatatatataatatataattaatatgtcagttaccacattttgttctttaaaaaaaacggacaagtgcaatatttatatttcaaaaagggctttctctgtgtttttattttatttgtttttttttttattcttccatttttattttctatgttcttcatcatttgctgttatcatacatgaaagtgaaaaaaggaaaaatatcatgagaaggaaaagatcaatactacaaatatttgaaaattctataaaatccaaagaaggaaaatgtaattttacaaaaagatatataactcattattataatatcccattaaaaatcaaaaaacatgacttacccagtgttataaaatatttttctcataaacctaatggaaagcataattatgttacaaatatgattacacaaaagaatagaaaatcgtttctatttttttttttcctatataataagtatttcttcggaaaacaagaacagataagaaaaatgaattatcatgaagaaatgaataaaataaatataaaaaatgatgggaatcgaaaaatatatatgtacccaaaaaatgacattcatgaagaggatggtgatcataagaatgatgtcgaaataaatcaaaaaaggaatgaacaaaattgtaaatcgtttaatgatgaaaaaaacgaaaatgctagagatccaaacaaaatattatatttgattaacccccgtggtttttgcaaaggtgttagtcgggctatagaaacggtagaagagtgcttaaaattatttaaaccacctatatatgtaaaacacaaaatagttcataacgatattgtttgtaaaaaattagagaaagaaggagcaatatttattgaagatttaaatgacgtacctgatggacatatattaatttattcagcacatggtattagtcctcaaatacgagaaatagcaaaaaaaaaaaaattaatagaaatagatgctacatgccctttagttaataaagtacatgtatatgtacaaatgaaagcaaaagaaaattatgacattattcttataggatataaaaatcatgtagaggttataggtacctataatgaagcaccacattgtacacatattgtggaaaatgttaatgatgtagataaattaaatttcccattaaataaaaagttattctatgttacacaaaccacactaagtatggatgattgtgcacttatcgtacaaaaactcaaaaataaattcccacatattgaaactatacctagtggatccatatgttatgctactacaaatagacaaacggctcttaataaaatatgtacaaaatgtgatcttaccatagttgttggtagttcttcatcttctaatgccaaaaaattagtctattcatcccaaatcagaaatgttccagcagtattacttaatacagtacatgatttagatcaacaaatacttaagaatgttaataaaatagcactaacttctgctgcctcaaccccagagcaagaaacacaaaaatttgtcaacctattaacaaaccctccatttaattataccttacaaaattttgacggggctcacgaaaatgtgcccaaatggaagcttcccaagaatttcttgcacatgataaaagaaagagaaaaatgaaatcacaaaaaaaaaaaaaaatatatatatatatatatatatatatatatatatatataaataaattagtgaaaaaaaaaaaatttttttttacattttgcacacaattta

SEQ ID NO: 2 - protein, 4 aa, Plasmodium falciparum
LYTY

SEQ ID NO: 3 - protein, 45 aa, Plasmodium falciparum
TKKKKKKKKKKKKKKLLTYIFYVLLYYLYFHLFIYLFYFIFFISR

SEQ ID NO: 4 - protein, 11 aa, Plasmodium falciparum
RYIYLYIYIYI

SEQ ID NO: 5 - protein, 539 aa, Plasmodium falciparum
YIINMSVTTFCSLKKTDKCNIYISKRAFSVFLFYLFFFLFFHFYFLCSSSFAVIIHESEKRKNIMRRKRSILQIFENSIKSKEGKCNFTKRYITHYYNIPLKIKKHDLPSVIKYFSHKPNGKHNYVTNMITQKNRKSFLFFFFLYNKYFFGKQEQIRKMNYHEEMNKINIKNDGNRKIYMYPKNDIHEEDGDHKNDVEINQKRNEQNCKSFNDEKNENARDPNKILYLINPRGFCKGVSRAIETVEECLKLFKPPIYVKHKIVHNDIVCKKLEKEGAIFIEDLNDVPDGHILIYSAHGISPQIREIAKKKKLIEIDATCPLVNKVHVYVQMKAKENYDIILIGYKNHVEVIGTYNEAPHCTHIVENVNDVDKLNFPLNKKLFYVTQTTLSMDDCALIVQKLKNKFPHIETIPSGSICYATTNRQTALNKICTKCDLTIVVGSSSSSNAKKLVYSSQIRNVPAVLLNTVHDLDQQILKNVNKIALTSAASTPEQETQKFVNLLTNPPFNYTLQNFDGAHENVPKWKLPKNFLHMIKEREK

SEQ ID NO: 6 - protein, 19 aa, Plasmodium falciparum
NHKKKKKYIYIYIYIYIYI

SEQ ID NO: 7 - protein, 13 aa, Plasmodium falciparum
KKKNFFLHFAHNL

SEQ ID NO: 8 - protein, 2 aa, Plasmodium falciparum
IN

SEQ ID NO: 9 - DNA, 1320 bp, Plasmodium falciparum
  gene (1)..(1320)
taaataaataaattataaatctttcaagaatatattttttataaaaacataaaatataaaatatacatatatatatatatatatattttatattacttttaaaattatttatttatacaaatggaaatttaatgtgaagaatagaaaaaacattttgtcaatatggaaaagtcaaaaaggtacataagcctgattaagatgatggaaaggaaaaaatttgagaagtatagattaaaacaaataatggataatatatataaaggaaaaataattgaaataaataaaatgaaaaatattccaactgaaataagaagagaattaaaaaatatatttcataataatattttaagtataaaaccgatcaaagaattaaaatatgatagagcatataaagtattatttcagtgtaaagataatgaaaagattgaagcaacatcattagattttggttcgcataaatctttatgtatatctagccaaataggttgttcttttggatgtaagttttgtgctactggtcaaattggtataaaaagacaattagatatagatgaaataactgatcaacttttatattttcaatcaaaaggagttgatataaaaaatatatcttttatgggtatgggagaacctttagctaatccatatgtttttgattctatacaattttttaatgataataatttattttctatatctaatagacgtattaatatatctactgttggtcttttaccaggaattaaaaaattaaataacatctttcctcaagttaatttagctttctcattacattctccatttactgaagaaagggatcaacttgtaccaattaataaattgtttccgtttaatgaagtttttgatttattagatgaaagaatagcaaaaactggtagaagagtttggataagttatattttaattaaaaatcttaatgactccaaagatcatgcagaagctttgtctgatcatatatgtaaaagaccaaataacataagatacttatataatgtatgtttaataccttataataaaggtaatagaatttataatatatcatttgaatatatatatatatatatatatttactaataataaaaaaaaagatattatgtaaatatattatgtttcacacattatataaatatataggcatagaggacatgttataaaaaagtgcaacatatatatatatatatatatatatatatatatatatacattttttttatatttatattatctttttaatacatttattccattacattgcagccaaaaatgttgacgaaaattttcatcgtttggacgatgct

SEQ ID NO: 10 - protein, 4 aa, Plasmodium falciparum
INKL

SEQ ID NO: 11 - protein, 27 aa, Plasmodium falciparum
IFQEYIFYKNIKYKIYIYIYIYILYYF

SEQ ID NO: 12 - protein, 9 aa, Plasmodium falciparum
NYLFIQMEI

SEQ ID NO: 13 - protein, 343 aa, Plasmodium falciparum
KKHFVNMEKSKRYISLIKMMERKKFEKYRLKQIMDNIYKGKIIEINKMKNIPTEIRRELKNIFHNNILSIKPIKELKYDRAYKVLFQCKDNEKIEATSLDFGSHKSLCISSQIGCSFGCKFCATGQIGIKRQLDIDEITDQLLYFQSKGVDIKNISFMGMGEPLANPYVFDSIQFFNDNNLFSISNRRINISTVGLLPGIKKLNNIFPQVNLAFSLHSPFTEERDQLVPINKLFPFNEVFDLLDERIAKTGRRVWISYILIKNLNDSKDHAEALSDHICKRPNNIRYLYNVCLIPYNKGNRIYNISFEYIYIYIYLLIIKKKILCKYIMFHTLYKYIGIEDML

SEQ ID NO: 14 - protein, 48 aa, Plasmodium falciparum
KSATYIYIYIYIYIYIHFFYIYIIFLIHLFHYIAAKNVDENFHRLDDA

SEQ ID NO: 15 - protein, 3 aa, Plasmodium falciparum
CEE

SEQ ID NO: 16 - DNA, 5 bp, Plasmodium falciparum
attta

SEQ ID NO: 17 - DNA, 37 bp, Plasmodium falciparum
atgtcagttaccacattttgttctttaaaaaaaacgg

SEQ ID NO: 18 - DNA, 31 bp, Plasmodium falciparum
gtgatttcatttttctctttcttttatcatc

SEQ ID NO: 19 - DNA, 21 bp, Plasmodium falciparum
aatctctgagcttcttctttg

SEQ ID NO: 20 - DNA, 31 bp, Plasmodium falciparum
gggggagctcgaacttaataaaaaagaggag

SEQ ID NO: 21 - DNA, 30 bp, Plasmodium falciparum
atggaaaagtcaaaaaggtacataagcctg

SEQ ID NO: 22 - DNA, 30 bp, Plasmodium falciparum
agcatcgtccaaacgatgaaaattttcgtc

SEQ ID NO: 23 - DNA, 21 bp, Plasmodium falciparum
aatctctgagcttcttctttg

SEQ ID NO: 24 - DNA, 31 bp, Plasmodium falciparum; identical to SEQ ID NO: 20

SEQ ID NO: 25 - DNA, 25 bp, Plasmodium falciparum
aggatcctccatcatcaaaccgaac

SEQ ID NO: 26 - DNA, 41 bp, Plasmodium falciparum
tcccatccactaaacttaaacatctattccggcctcgttat

SEQ ID NO: 27 - DNA, 41 bp, Plasmodium falciparum
atgtttaagtttagtggatgggaagcggtctgatagccatt

SEQ ID NO: 28 - DNA, 25 bp, Plasmodium falciparum
agtcgacaagtggagcctgcttttc